(19) United States
(12) Patent Application Publication
(10) Pub. No.: US 2006/0041303 A1
(43) Pub. Date: Feb. 23, 2006

(54) Guidewire with stopper

(76) Inventor: Henry M. Israel, Bnei Brak (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 10/920,241

(22) Filed: Aug. 18, 2004

(51) Int. Cl. A61F 2/06

(52) U.S. Cl. 623/1.11

(57) ABSTRACT

A stent assembly including a catheter formed with a side aperture, and a branch guidewire disposed in the catheter and which passes into the side aperture, the branch guidewire including a stopper sized such that the stopper cannot be pulled through the side aperture into the catheter but can be fixedly held in the side aperture and pushed outwards from the side aperture.

### FIELD OF THE INVENTION

The present invention relates generally to stents, and particularly to a guidewire with a stopper that facilitates implanting bifurcated stents in a body.

### BACKGROUND OF THE INVENTION

A stent is a well known device used to support an intraluminal wall, used in procedures, such as but not limited to, percutaneous transluminal coronary angioplasty (PTCA). Various types of stent architectures are known in the art, including braided stents (filaments or wires, wound or braided into a particular configuration), or mesh stents (metal mesh bent or formed into a particular shape), among others.

Typically, a stent may be restrained in a radially compressed configuration by a sheath or catheter, and delivered by an introducer to the site where it is required. The introducer may pass over a guidewire (like a monorail) that has been entered through the patient's skin, or through a blood vessel exposed by minor surgical means. When the introducer has been threaded into the body lumen to the stent deployment location, the introducer is manipulated to cause the stent to be released. The stent expands to a predetermined diameter at the deployment location, and the introducer is withdrawn. Stent expansion may be effected by spring elasticity, balloon expansion, or by the self-expansion of a thermally or stress-induced return of a shape memory alloy (such as a nickel-titanium alloy, e.g., NITINOL) to a pre-conditioned expanded configuration.

There are bifurcated lumens, such as but not limited to, the carotid artery, which may need support with a bifurcated stent. A bifurcated lumen (also called bifurcation) is an area of the vasculature where a first vessel is bifurcated into two or more branch vessels. Stenotic lesions may form in or around such bifurcations, that is, in or around one or more of the vessels.

However, delivering and deploying a stent to support a bifurcated lumen is a difficult challenge. Some of the problems include the difficulty of properly orienting the stent with respect to the bifurcation and the difficulty of providing a stent that supports the main trunk and branches of the bifurcation without blocking the passageways or causing turbulence or other flow disruptions.

PCT patent application PCT/IL03/00814 to Henry Israel, describes a bifurcated stent assembly with a stent sheath that includes two individually removable portions, one removable in a distal direction and the other in a proximal direction. Binds and you idiot the stent can also be over the catheter and get ballooned

U.S. Pat. No. 6,494,905 to Zedler et al. describes a balloon catheter for use in the region of a vessel branching and, in particular, in coronary vessels. The catheter comprising a catheter stem disposed at the distal end of which are provided at least one balloon and at least one first guide means arranged in the region thereof for positioning the balloon in the region of a vessel branching. The first guide means is adapted to be introduced into the lateral branch of the vessel transversely with respect to the longitudinal direction of the balloon catheter. The balloon comprises at least two chambers which are spaced from each other in the longitudinal direction of the balloon catheter and between which the first guide means is arranged

U.S. Pat. No. 6,761,734 to Suhr describes a segmented balloon catheter for use in treating a condition of a blood vessel occurring near a bifurcation. The catheter comprises a shaft which includes a proximal end, a distal end and a longitudinal passageway that extends therethrough from the proximal end to the distal end. A first balloon portion is mounted on the shaft adjacent the distal end, and a second balloon portion is mounted on the shaft adjacent the first balloon portion. The shaft also comprises a transverse port which extends between the longitudinal passageway and the exterior of the segmented balloon catheter from between the first and second balloon portions. In this manner, a proximal end of a first guide wire which is pre-positioned in the main vessel may be inserted into the distal end of the shaft and threaded through the longitudinal passageway and out the proximal end of the shaft, and a proximal end of a second guide wire which is pre-positioned in the side branch vessel may be inserted into the transverse port and threaded through the longitudinal passageway and out the proximal end of the shaft.

A problem of the prior art stent assemblies is that the stent/catheter must be slid over a double guidewire (i.e., a pair of guidewires). At some point along the travel over the guidewires, the stent/catheter tends to twist and bind or otherwise get snagged, hindering the travel of the stent to the desired site.

### SUMMARY OF THE INVENTION

The present invention seeks to provide a guidewire that facilitates implanting bifurcated stents in a body, and which may be useful in deploying a guidewire through a side aperture of a main stent into a bifurcation of a blood vessel, for example. The guidewire of the present invention permits introducing a branch stent over a single guidewire and obviates the need for trying to push a guidewire through the side aperture.

There is provided in accordance with an embodiment of the present invention a stent assembly including a catheter formed with a side aperture, and a branch guidewire disposed in the catheter and which passes into the side aperture, the branch guidewire including a stopper sized such that the stopper cannot be pulled through the side aperture into the catheter but can be fixedly held in the side aperture and pushed outwards from the side aperture. The stopper may be positioned at a distal portion of the guidewire. The stopper may be initially fixedly held in the side aperture of the catheter. The stopper may have a spherical shape.

In accordance with an embodiment of the present invention the catheter may be slid over a main guidewire. A main stent may be assembled with the catheter. The branch guidewire may pass through the side aperture of the catheter and through a side opening of the main stent. A branch stent may slide over the branch guidewire and pass through the side opening of the main stent.

In accordance with another embodiment of the present invention two or more inflatable chambers may be disposed longitudinally along the catheter, with the side aperture between the inflatable chambers.

### BRIEF DESCRIPTION OF DRAWINGS

The present invention will be further understood and appreciated from the following detailed description taken in conjunction with the drawing in which:

FIG. 1 is a simplified pictorial illustration of a stent assembly, constructed and operative in accordance with an embodiment of the invention, including a catheter introduced into a body lumen, such as near a bifurcation, and including a branch guidewire with a stopper fixedly held in a side aperture of the catheter;

FIG. 2 is a simplified illustration of the stopper being pushed into a branch lumen of the bifurcation;

FIG. 3 is a simplified illustration of introducing a branch stent over the branch guidewire and through the side aperture, in accordance with an embodiment of the invention; and

FIG. 4 is a simplified pictorial illustration of a stent assembly, constructed and operative in accordance with another embodiment of the invention, with the side aperture between two or more inflatable chambers.

### DETAILED DESCRIPTION OF EMBODIMENTS

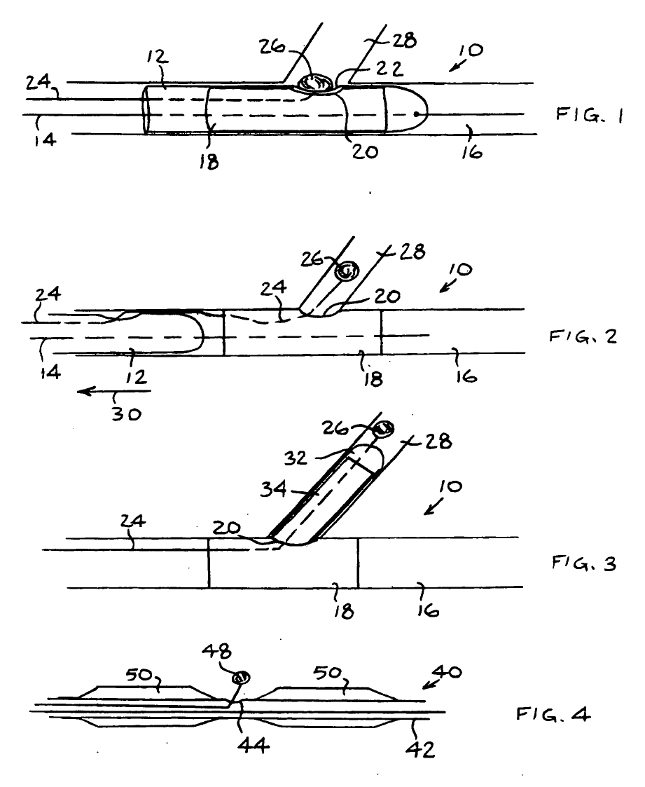

Reference is now made to FIG. 1, which illustrates a stent assembly 10, constructed and operative in accordance with an embodiment of the invention. Stent assembly 10 may include a catheter 12, which may be passed over a main guidewire 14 to enter a body lumen 16, such as but not limited to a blood vessel. As is well known in angioplasty, the main guidewire 14 may be introduced by means of an introducer sheath (not shown) inserted through some lumen (e.g., femoral artery), and the guidewire 14 is manipulated through vasculature to the site of implanting the stent.

A main stent 18 may be initially disposed in catheter 12 in a contracted orientation prior to deployment, and slides over main guidewire 14 together with catheter 12. In such an embodiment, the main stent 18 is a self-expanding stent, which may be constructed from a suitable material, such as but not limited to, a shape memory alloy (such as a nickel-titanium alloy, e.g., NITINOL). Main stent 18 may be a wire mesh or braided stent, or any other kind of stent, but the invention is not limited to this construction or to self-expanding stents. For example, without limitation, stent 18 may be balloon-expandable, constructed from a suitable material, such as but not limited to, stainless steel 316L. In such an embodiment, the stent 18 is disposed over the catheter 12. In general, stent 18 is “assembled with” the catheter 12, which encompasses self-expanding, balloon-expandable and any other kinds of stents. Stent 18 may be coated, such as a drug-eluting stent that has a polymer coating that emits an anti-restenosis drug. Stent 18 may be formed with a side opening 20 and catheter 12 may be formed with a side aperture 22 for placing therethrough a branch stent, as is described hereinbelow.

Another guidewire 24, referred to as a branch guidewire 24, may be disposed in catheter 12. The branch guidewire 24 passes through both the side opening 20 of main stent 18 and the side aperture 22 of catheter 12. Branch guidewire 24 may include a stopper 26 at a distal portion thereof (“distal portion” refers to the distal tip or proximal to, that is, set back from, the distal tip). Stopper 26 may be a ball (sphere) or another shape attached to or integrally formed with branch guidewire 24. Stopper 26 may be sized such that it cannot be pulled through the side aperture 22 into the catheter 12 but can be fixedly held in the side aperture 22 and pushed outwards from the side aperture 22. Stopper 26 may indeed be initially fixedly held in the side aperture 22 of catheter 12. The guidewires 14 and 24 may be grasped and manipulated (e.g., pushed, pulled, twirled and twisted) at the proximal ends thereof, as is well known in the art. The guidewires 14 and 24 may be constructed of any suitable material for guidewires, as is well known in the art.

FIG. 1 illustrates catheter 12 after being slid over main guidewire 14 to the site of a bifurcation. The main stent 18 is positioned at the bifurcation and the branch guidewire 24 is aligned with a branch lumen 28 of the bifurcation such that the stopper 26 (and possibly some of the branch guidewire 24) enters the branch lumen 28.

Reference is now made to FIG. 2. Catheter 12 may be moved off main stent 18 so that main stent 18 may expand or be otherwise deployed at the bifurcation The branch guidewire 24 may be pushed into the branch lumen 28. The branch guidewire 24 together with stopper 26 may be manually held in branch lumen 28 while the catheter 12 is withdrawn proximally out of the body lumen 16 in the direction of arrow 30.

Reference is now made to FIG. 3. Another catheter 32 may be slid over branch guidewire 24. A branch stent 34 may be initially disposed in catheter 32 in a contracted orientation prior to deployment, and slides over branch guidewire 24 together with catheter 32. As described hereinabove for main stent 18, branch stent 32 may be a wire mesh or braided stent, or any other kind of stent, e.g., balloon-expandable or self-expanding, and may be coated, such as a drug-eluting stent. The branch stent 34 may be introduced through the side opening 20 of main stent 18 into the branch lumen 28. Upon withdrawal of catheter 32, branch stent 34 may expand or be otherwise deployed in branch lumen 28.

It is noted that the terms “push” and “pull” are relative terms and encompass any suitable motion and are not limited to the strict sense of push or pull. Also, the terminology of “sliding” a catheter over a guidewire is meant to encompass any kind of motion of the catheter with respect to the guidewire, such as but not limited to, monorail fashion.

Reference is now made to FIG. 4, which illustrates a stent assembly 40, constructed and operative in accordance with another embodiment of the invention. This embodiment may include a balloon catheter 42, which may be similar in construction to those shown in U.S. Pat. No. 6,494,905 to Zedler et al. or U.S. Pat. No. 6,761,734 to Suhr. As in the previously described embodiments, catheter 42 may be formed with a side aperture 44. A branch guidewire 46 may be disposed in catheter 42 and passes into side aperture 44. Branch guidewire 46 includes a stopper 48 sized such that the stopper 48 cannot be pulled through the side aperture 44 into the catheter 42 but can be fixedly held in the side aperture 44 and pushed outwards from the side aperture 44. Two or more inflatable chambers 50 may be disposed longitudinally along the catheter 42, and the side aperture 44 is between the inflatable chambers 50. The operation of the stent assembly 40 is basically as described hereinabove for stent assembly 10.

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

What is claimed is:

1. A stent assembly comprising:
a catheter formed with a side aperture; and
a branch guidewire disposed in said catheter and which passes into said side aperture, said branch guidewire comprising a stopper sized such that the stopper cannot be pulled through the side aperture into the catheter but can be fixedly held in the side aperture and pushed outwards from the side aperture.

2. The stent assembly according to claim 1, wherein said stopper is positioned at a distal portion of the guidewire.

3. The stent assembly according to claim 1, wherein said stopper is initially fixedly held in the side aperture of said catheter.

4. The stent assembly according to claim 1, wherein said stopper has a spherical shape.

5. The stent assembly according to claim 1, further comprising a main guidewire over which said catheter is slidable.

6. The stent assembly according to claim 5, further comprising a main stent assembled with said catheter.

7. The stent assembly according to claim 6, wherein said branch guidewire passes through the side aperture of said catheter and through a side opening of said main stent.

8. The stent assembly according to claim 1, further comprising a branch stent that slides over said branch guidewire.

9. The stent assembly according to claim 7, further comprising a branch stent that slides over said branch guidewire and passes through said side opening of said main stent.

10. The stent assembly according to claim 1, further comprising at least two inflatable chambers disposed longitudinally along the catheter, said side aperture being between said inflatable chambers.
